(19) United States
(12) Patent Application Publication
(10) Pub. No.: US 2026/0071172 A1
(43) Pub. Date: Mar. 12, 2026

(54) METHOD FOR ENHANCING DEGRADATION PERFORMANCE OF LIGNIN-DEGRADING BACTERIA

(71) Applicant: SHENZHEN RESEARCH INSTITUTE OF NORTHWEST A & F UNIVERSITY, Shenzhen (CN)

(72) Inventors: Yahong WEI, Shenzhen (CN); Lili HUANG, Shenzhen (CN); Shuting ZHAO, Shenzhen (CN); Dongtao DENG, Shenzhen (CN); Lei DENG, Shenzhen (CN); Qianyi TIAN, Shenzhen (CN); Jie FENG, Shenzhen (CN); Yingjia ZHAO, Shenzhen (CN)

(73) Assignee: SHENZHEN RESEARCH INSTITUTE OF NORTHWEST A & F UNIVERSITY, Shenzhen (CN)

(21) Appl. No.: 19/389,022

(22) Filed: Nov. 14, 2025

Related U.S. Application Data
(63) Continuation of application No. PCT/CN2024/072354, filed on Jan. 15, 2024.

(30) Foreign Application Priority Data
May 15, 2023 (CN) 202310542732.2

(51) Int. Cl. C12N 1/20; C12N 9/02; C12N 9/08; C12R 1/18

(52) U.S. Cl. CPC C12N 1/205; C12N 9/0061; C12N 9/0065; C12R 2001/18; C12Y 110/03002; C12Y 111/01013; C12Y 111/01014

(57) ABSTRACT

A method for enhancing the degradation performance of lignin-degrading bacteria Erwinia sp. QL-Z3 and a culture medium for culturing the bacteria. The rate of degradation of an Erwinia sp. QL-Z3 strain to lignin is optimized from 14.23% before optimization to 25.01%. Under the conditions that the initial pH value of the culture medium is 8, the nitrogen source is NH₄NO₃, and the addition amount of lignin is 3 g/L, the activity of an LiP enzyme can be optimized to 371.00 U/L, which is 3.53 times that before optimization. When the initial pH value of the culture medium is 9.5, the nitrogen source is NH₄NO₃, and the concentration of lignin is 2.5 g/L, the activity of MnP and Lac enzymes can be optimized to 839.50 U/L and 219.00 U/L, respectively, which are 3.18 and 2.84 times that before optimization.

### CROSS-REFERENCE TO RELATED APPLICATION

This application is a continuation application of International Application No. PCT/CN2024/072354, filed on Jan. 15, 2024, which claims priority to Chinese Parent application No. 202310542732.2, filed on May 15, 2023, the entire contents of all of which are incorporated herein by reference.

### TECHNICAL FIELD

The present disclosure relates to the field of microbiological technology and refers to the fermentation culture of microorganisms, specifically to a method for enhancing the degradation performance of lignin degradation bacteria.

### BACKGROUND

Lignocellulose mainly consists of cellulose, hemicellulose and lignin. Lignin is a high-molecular polymer composed of phenylpropane derivatives, exists in the xylem of plants and is the most abundant aromatic compound in nature. Lignin and its products can be used to prepare high-value chemicals such as vanillin and ferulic acid, and lignin derivatives have great potential in applications such as sensor components, bio-composites, biofuels, hydrogels and bulk chemicals.

Biorefineries and the pulp and paper industry produce a large amount of papermaking black liquor, which contains a large amount of lignin difficult to degrade. It is reported that the pulp industry alone produces about 50 million tons of lignin annually, and only about 2% of the lignin in the paper industry is used for commercial purposes, while the rest is directly discharged or incinerated. This not only leads to the underutilization of lignin resources but also causes serious pollution to the natural environment. In addition, lignin, as a part of straw, is difficult to naturally degrade even through straw returning to the field. Therefore, how to accelerate the degradation of lignin in wastewater and waste straw through biotechnology and convert it into energy, chemical products, etc., not only helps to control lignin pollution but also plays a role in the comprehensive utilization of biomass energy.

### SUMMARY

The purpose of the present disclosure is to improve the degradation efficiency of lignin by lignin degradation bacteria and simultaneously enhance the enzymatic activity of enzymes closely related to lignin degradation by optimizing the culture and fermentation conditions of lignin degradation bacteria.

To achieve the technical purpose of the present disclosure, the technical solution of the present disclosure focuses on the lignin degradation bacteria Erwinia sp. QL-Z3. The GenBank accession number of 16S rRNA gene in the Erwinia sp. QL-Z3 strain is MH828331, and the GenBank accession number of the whole gene sequencing is CP037950.

Specifically, the present disclosure provides a method for enhancing degradation performance of lignin degradation bacteria, the lignin degradation bacteria are Erwinia sp. QL-Z3 strain, and activated Erwinia sp. QL-Z3 strain are inoculated into lignin liquid medium at 30° C. and 180 rpm for cultivation.

The pH of the lignin liquid medium is 5, nitrogen source is (NH₄)₂SO₄, and lignin addition amount is 1.5 g/L.

Components and contents of each 1 L of the lignin liquid medium are: 1.5 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of (NH₄)₂SO₄.

The disclosure optimizes the optimal fermentation conditions of the Erwinia sp. QL-Z3 strain, and under this conditions, degradation rate of the lignin by the Erwinia sp. QL-Z3 strain is optimized to 25.24%.

In some preferred embodiments of the method for enhancing the degradation performance of lignin degradation bacteria, by adjusting pH of the lignin liquid medium to 8, nitrogen source to NH₄NO₃, and lignin addition amount to 3 g/L, enzyme activity of the lignin peroxidase in the Erwinia sp. QL-Z3 strain is optimized to 371.00 U/L.

In some preferred embodiments of the method for enhancing the degradation performance of lignin degradation bacteria, by adjusting pH of the lignin liquid medium to 9.5, the nitrogen source to NH₄NO₃, and the lignin addition amount to 2.5 g/L, the enzyme activity of the laccase in the Erwinia sp. QL-Z3 strain is optimized to 219.00 U/L.

In some preferred embodiments of the method for enhancing the degradation performance of lignin degradation bacteria, by adjusting the pH of the lignin liquid medium to 9.5, the nitrogen source to NH₄NO₃, and the lignin addition amount to 2.5 g/L, the enzyme activity of the manganese peroxidase in the Erwinia sp. QL-Z3 strain is optimized to 839.50 U/L.

The present disclosure further provides a medium for enhancing degradation performance of lignin degradation bacteria, where the medium is a lignin liquid medium, pH of the lignin liquid medium is 5, nitrogen source is (NH₄)₂SO₄, and lignin addition amount is 1.5 g/L; components and contents of each 1 L of the lignin liquid medium are: 1.5 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of (NH₄)₂SO₄; Erwinia sp. QL-Z3 strain of the lignin degradation bacteria are cultivated in the lignin liquid medium at 30° C. and 180 rpm, and lignin degradation rate of the Erwinia sp. QL-Z3 strain is optimized to 25.24%.

The present disclosure further provides a medium for enhancing enzyme activity of lignin peroxidase in lignin degradation bacteria, wherein the medium is a lignin liquid medium, pH of the lignin liquid medium is 8, nitrogen source is NH₄NO₃, and lignin addition amount is 3 g/L; components and contents of each 1 L of the lignin liquid medium are: 3 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of NH₄NO₃, Erwinia sp. QL-Z3 strain of the lignin degradation bacteria are cultivated in the lignin liquid medium at 30° C. and 180 rpm, and the enzyme activity of the lignin peroxidase in the Erwinia sp. QL-Z3 strain is optimized to 371.00 U/L.

The present disclosure further provides a medium for enhancing enzymatic activity of laccase in lignin degradation bacteria, wherein the medium is a lignin liquid medium, with a pH of 9.5, nitrogen source of NH₄NO₃, and lignin addition of 2.5 g/L; components and contents of each 1 L of the lignin liquid medium are: 2.5 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of NH₄NO₃. Erwinia sp. QL-Z3 strain of the lignin degradation bacteria are cultivated in the lignin liquid medium at 30° C. and 180 rpm, and the enzymatic activity of the laccase in the Erwinia sp. QL-Z3 strain is optimized to 219.00 U/L.

The present disclosure further provides a medium for enhancing enzymatic activity of manganese peroxidase in lignin degradation bacteria, wherein the medium is a lignin liquid medium, with a pH of 9.5, nitrogen source of NH₄NO₃, and lignin addition of 2.5 g/L; the composition and content of each 1 L of the lignin liquid medium are: 2.5 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of NH₄NO₃; Erwinia sp. QL-Z3 strain of the lignin degradation bacteria are cultivated in the lignin liquid medium at 30° C. and 180 rpm, and the enzymatic activity of manganese peroxidase in Erwinia sp. QL-Z3 strain are optimized to 839.50 U/L.

Compared with the existing technologies, the disclosure “method for enhancing the degradation Performance of lignin degradation bacteria” has the following beneficial effects:

The degradation of lignin by microorganisms is a process of converting large lignin molecules into small molecules and being absorbed by microbial cells, which is a result of multiple enzymes secreting and functioning during the strain fermentation process. The disclosure optimizes the degradation rate and lignin degradation enzyme activity of the Erwinia sp. QL-Z3 strain to find its optimal conditions, thereby maximizing the lignin degradation ability of the Erwinia sp. QL-Z3 strain.

The optimal conditions for the fermentation and degradation of lignin by the Erwinia sp. QL-Z3 strain are: the initial pH of the culture medium is 5.0, the nitrogen source is (NH₄)₂SO₄, and the lignin concentration is 1.5 g/L. Under these conditions, the lignin degradation rate of the Erwinia sp. QL-Z3 strain is optimized from 14.23% to 25.01%, with a significant optimization result.

The single-factor optimization and orthogonal experiment optimization of the LiP, Lac, and MnP enzyme activities in the supernatant of the Erwinia sp. QL-Z3 strain fermentation were carried out. Under the conditions of pH 8, nitrogen source NH₄NO₃, and lignin addition of 3 g/L, the enzyme activity of the LiP crude enzyme solution can be optimized to 371.00 U/L, which is 3.53 times that of before optimization, with a significant optimization result. The optimal fermentation conditions for enzyme activities of the MnP and Lac crude enzyme solution are: the initial pH of the culture medium is 9.5, the nitrogen source is NH₄NO₃, and the lignin concentration is 2.5 g/L. Under these conditions, the manganese peroxidase enzyme activity of the Erwinia sp. QL-Z3 strain is 839.50 U/L, and that of the laccase is 219.00 U/L, which are 3.18 and 2.84 times that of before optimization, respectively, with a significant optimization result.

### ILLUSTRATED DESCRIPTION

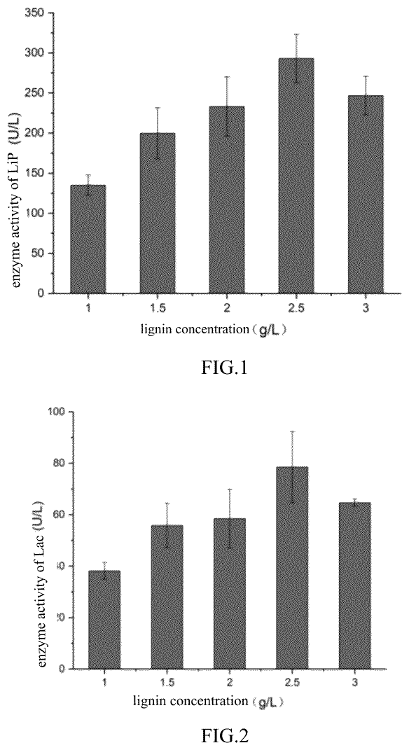

FIG. 1 shows the influence of different lignin concentrations on the enzymatic activity of lignin peroxidase (LiP).

FIG. 2 shows the influence of different lignin concentrations on the enzymatic activity of laccase (Lac).

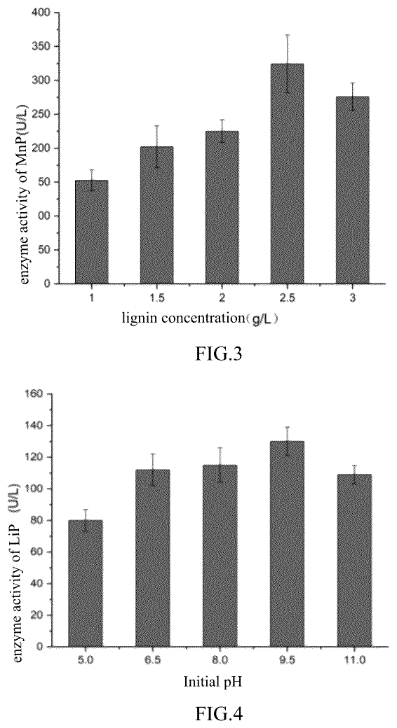

FIG. 3 shows the influence of different lignin concentrations on the enzymatic activity of manganese peroxidase (MnP).

FIG. 4 shows the influence of different pH values on the enzymatic activity of lignin peroxidase (LiP).

FIG. 5 shows the influence of different pH values on the enzymatic activity of laccase (Lac).

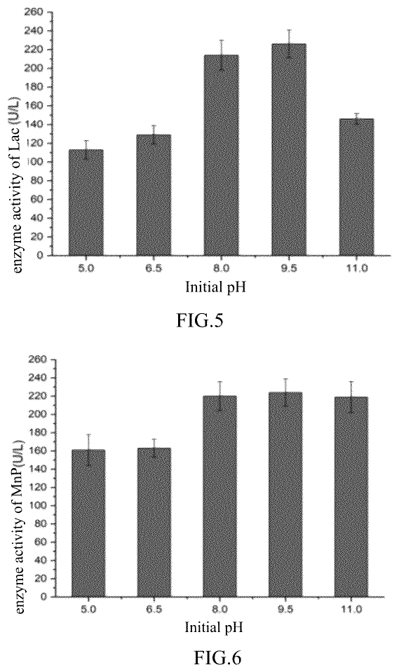

FIG. 6 shows the influence of different pH values on the enzymatic activity of manganese peroxidase (MnP).

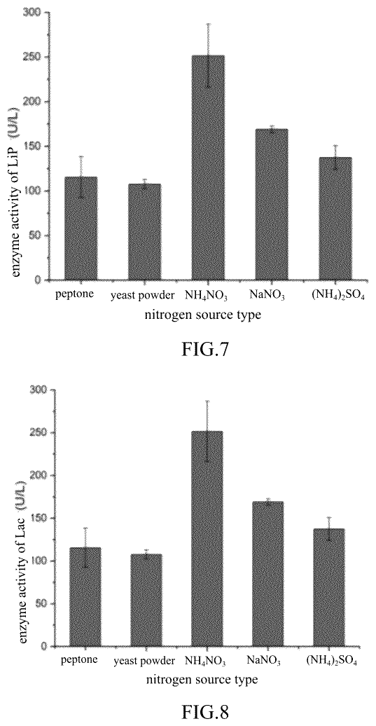

FIG. 7 shows the influence of different nitrogen source types on the enzymatic activity of lignin peroxidase (LiP).

FIG. 8 shows the influence of different nitrogen source types on the enzymatic activity of laccase (Lac).

FIG. 9 shows the influence of different nitrogen source types on the enzymatic activity of manganese peroxidase (MnP).

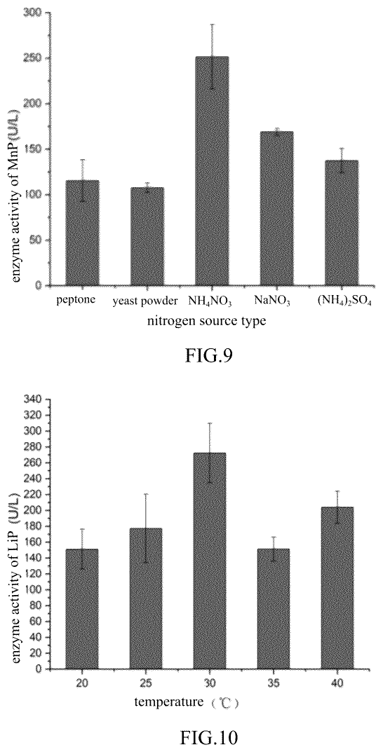

FIG. 10 shows the influence of different temperatures on the enzymatic activity of lignin peroxidase (LiP).

FIG. 11 shows the influence of different temperatures on the enzymatic activity of laccase (Lac).

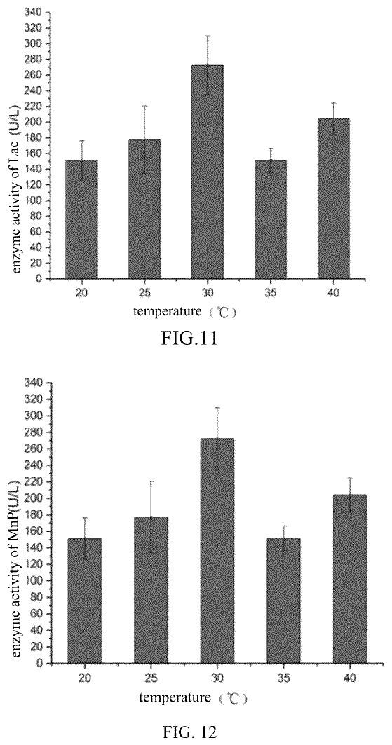

FIG. 12 shows the influence of different temperatures on the enzymatic activity of manganese peroxidase (MnP).

### DETAILED DESCRIPTION

The disclosure is explained based on the examples as below. The technical solutions in the embodiments of the disclosure are described clearly and completely. Obviously, the described embodiments are only a part of the embodiments of the disclosure, rather than all the embodiments. Based on the embodiments of the disclosure, all other embodiments obtained by ordinary skilled technicians in the field without making creative efforts fall within the protection scope of the disclosure.

The culture medium used in the embodiments is shown in Table 1.

TABLE 1
Formulas of Used Culture Medium

| Name | Formulas |
|---|---|
| Lignin liquid | alkali lignin 2 g, K₂HPO₄ 2 g, MgSO₄•7H₂O 0.3 g, CaCl₂ |
| culture medium | 0.08 g, FeSO₄•7H₂O 0.05 g, MnCl₂ 0.02 g, (NH₄)₂SO₄ 2 g, |
| (1 L) | pH value 7.0~7.2 |
| LB culture medium | peptone 10 g, yeast powder 5 g, NaCl 10 g, pH value |
| (1 L) | 7.0~7.2 |
| Glucose culture | glucose 2 g, K₂HPO₄ 2 g, MgSO₄•7H₂O 0.3 g, CaCl₂ |
| medium (1 L) | 0.08 g, FeSO₄•7H₂O 0.05 g, MnCl₂ 0.02 g, (NH₄)₂SO₄ 2 g, |
| | pH value 7.0~7.2 |

The enzyme activity determination methods in the examples are as follows.

Lignin peroxidase: in the reaction system of 3 mL, the reaction mixture contains 1.85 mL of 0.24 mmol/L resveratrol and 1.0 mL of crude enzyme solution and is preheated to 37° C., then 0.1 mL of 6.0 mmol/L H₂O₂ is added to initiate the reaction, and the increase of absorbance value at 310 nm before and after 3 minutes is measured. One enzyme activity unit is represented as the amount of enzyme that increasing the absorbance value by 0.1 per minute.

Laccase: in a reaction system of 3 mL in 25° C., the reaction mixture contains 2 mL of 0.5 mmol/L ABTS (dissolved in acetic acid-acetate sodium buffer solution of 0.1 mmol/L with pH 5.0), and 1 mL of crude enzyme solution is added to initiate the reaction. The absorbance value at 420 nm is measured every 1 minute, and part of linear change thereof is taken. One enzyme activity unit is represented as the amount of enzyme that increasing the absorbance value by 0.1 per minute.

Manganese peroxidase: in a reaction system of 3 mL in 37° C., the reaction mixture contains 2.4 mL of 50 mmol/L acetic acid buffer with pH 4.5, 0.1 mL of 1.6 mmol/L MnSO₄ solution, and 0.4 mL of crude enzyme solution. 0.1 mL of 1.6 mmol/L H₂O₂ solution is added to initiate the reaction at 37° C. The absorbance value at 240 nm is measured initially within the first 3 minutes and part of linear change thereof is taken. One enzyme activity unit is represented as the amount of enzyme that increasing the absorbance value by 0.1 per minute.

The methods for determining the lignin degradation rate in the examples are as follows.

The QL-Z3 strain is inoculated into LB liquid medium for activation culture until the OD600 is about 0.9. The obtained solution is centrifuged at 5000 rpm for 5 minutes, and after discarding the supernatant, the obtained material is washed twice with sterile water and inoculated by 1% inoculation amount into the lignin liquid medium, then is placed in a constant temperature shaker for shake flask culture at 30° C. and 180 rpm. After 3 days of cultivation, the fermentation broth of the lignin shake flask culture is collected for centrifuging at 8000 rpm for 5 minutes and concentrating. The upper crude enzyme solution is filtered by a 0.22 μm microporous water filter membrane to sterilize and then OD value thereof is measured at 280 nm. The lignin degradation rate is calculated based on the lignin standard curve. The formula is as follows:

$$Degradation rate = {( {A}_{0} - A )}_{n} / {A}_{0} \times 100 \%$$

Where, A₀ is the concentration of lignin in the culture medium before inoculation; An is the concentration of lignin in the culture medium after n hours of inoculation.

### Example 1

This example provides three levels for three influencing factors namely initial pH of culture medium (A), nitrogen source type (B), and lignin concentration (C), to optimize the lignin degradation rate of strain QL-Z3 through orthogonal experiments. Table 2 shows the factors to be measured and their corresponding influencing levels. The experimental design table is shown in Table 3.

TABLE 2
Factors and Levels Required for Experimental Design

| | Factors | | |
|---|---|---|---|
| | pH of culture | Nitrogen Source | Additive Amount of Lignin |
| Level | medium(A) | Type(B) | g · L⁻¹(C) |
| 1 | 5 | NaNO₃ | 1 |
| 2 | 7 | (NH₄)₂SO₄ | 1.5 |
| 3 | 9 | Peptone | 2 |

TABLE 3
Experimental Design Table

| Test | Initial | Nitrogen | Additive Amount of | Lignin Degradation |
|---|---|---|---|---|
| Number | pH(A) | Source(B) | Lignin (g · L⁻¹)(C) | Rate % |
| 1 | 1(5) | 2((NH₄)₂SO₄) | 2(1.5) | 25.24 |
| 2 | 1(5) | 3(Peptone) | 3(2) | 6.7 |
| 3 | 1(5) | 1(NaNO₃) | 1(1) | 12.6 |
| 4 | 2(7) | 2((NH₄)₂SO₄) | 1(1) | 11.4 |
| 5 | 2(7) | 1(NaNO3) | 3(2) | 4.59 |
| 6 | 2(7) | 3(Peptone) | 2(1.5) | 19.64 |
| 7 | 3(9) | 1(NaNO₃) | 2(1.5) | 10.63 |
| 8 | 3(9) | 3(Peptone) | 1(1) | 3.03 |
| 9 | 3(9) | 2((NH₄)₂SO₄) | 3(2) | 8.5 |
| K₁ | 44.54 | 27.82 | 27.03 | |
| K₂ | 35.63 | 45.14 | 55.51 | |
| K₃ | 22.16 | 29.37 | 19.79 | |
| k₁ | 14.85 | 9.27 | 9.01 | |
| k₂ | 11.88 | 15.05 | 18.5 | |
| k₃ | 7.39 | 9.79 | 6.6 | |
| Range R | 7.46 | 5.78 | 11.9 | |
| Optimal level | A₁ | B₂ | C₂ | |
| Primary and | C > A > B | | | |
| secondary | | | | |
| levels | | | | |
| Optimal | A₁B₂C₂ | | | |
| combination | | | | |

Note:
K₁, K₂, and K₃ are the sums of the degradation rates at levels 1, 2, and 3 respectively under a single factor, k₁, k₂, and k₃ are the corresponding average values, and R is the difference between the maximum and minimum k values.

TABLE 4
Orthogonal Model Variance Analysis

| | | Degrees | | | | |
|---|---|---|---|---|---|---|
| | Sum of | of | Mean | | | |
| Source | Squares | Freedom | Square | F Value | P Value | Significance |
| Correction | 329.643^{a} | 6 | 54.940 | 21.026 | 0.033 | * |
| model | | | | | | |
| Intercept | 986.588 | 1 | 986.588 | 536.938 | 0.002 | ** |
| A | 137.144 | 2 | 68.572 | 37.320 | 0.026 | * |
| B | 101.276 | 2 | 50.638 | 27.559 | 0.035 | * |
| C | 91.222 | 2 | 45.611 | 24.823 | 0.039 | * |
| Error | 3.675 | 2 | 1.837 | | | |
| Total | 1319.906 | 9 | | | | |
| Corrected | 313.318 | 8 | | | | |
| Total | | | | | | |
| Correlation | R² = 0.989(Adjusted to R² = 0.956) | | | | | |
| Coefficient | | | | | | |

Note:
** represents p < 0.01, indicating extremely significant difference;
* represents p < 0.05, indicating significant difference.

From Table 4, it can be seen that F value of the model is 21.026, and P value is less than 0.05, R²=0.989 is close to 1, which indicates that the reliability of this result is high, the model is significant, and can be used to predict the effects of the three factors namely initial pH, nitrogen source, and lignin concentration on the degradation rate. The results of the orthogonal combination variance model analysis show that the effects of the initial pH (A), the nitrogen source (B), and the lignin concentration (C) on the degradation of lignin by the strain are significant. Based on the R values corresponding to each factor, the influence of each factor on the degradation situation can be determined. The larger the R value, the stronger the influence on the result, and the ranking is: lignin concentration (C)>initial pH of the culture medium (A)>nitrogen source type (B).

Through experimental analysis, A₁B₂C₂ is the optimal fermentation condition combination for QL-Z3 to degrade lignin, that is, pH is selected as 5, the nitrogen source is (NH₄)₂SO₄, the lignin concentration is 1.5 g/L, and the final degradation rate can be optimized to 25.24%. The lignin degradation rate results of four verification experiments for this optimal combination are: 25.24%, 24.28%, 24.89%, 25.67%. The average degradation rate after repetition is 25.01%, the experimental results have repeatability, and thus the experimental results are valid.

### Example 2

This example provides the influence of lignin concentration on the enzyme activity of lignin peroxidase (LiP), laccase (Lac), and manganese peroxidase (MnP) produced by the strain QL-Z3.

The strain QL-Z3 was inoculated into 50 mL of liquid LB medium (containing 50 mg/L ampicillin), cultured at 30° C. and 180 rpm for overnight. The bacterial solution was transferred to 2 mL EP tubes for freezing centrifugation and concentration, then was washed by sterilized saline water to remove the remaining LB, and suspended. Then, the bacterial suspension was inoculated into different lignin liquid culture medium with different initial concentrations (1.0, 1.5, 2.0, 2.5, and 3.0 g/L), with 3 replicates for each concentration gradient. The culture medium was cultivated at 30° C. and 180 rpm for 7 days, and the fermentation was centrifuged and the supernatant was collected every 24 hours to measure the enzyme activity of LiP, Lac, and MnP, and the enzyme activity change curves were plotted.

From FIGS. 1, 2, and 3, it can be seen that between 1 g/L and 2 g/L lignin concentration, the enzyme activities of lignin peroxidase (LiP), laccase (Lac), and manganese peroxidase (MnP) continuously increase, and the change trend is consistent. When the lignin concentration changes from 2.0 g/L to 2.5 g/L, the three enzymes increase significantly with the increase of lignin concentration and reach the maximum enzyme activity. The optimal lignin concentration is 2.5 g/L, and the enzyme activities of LiP, Lac, and MnP are: 293.00 U/L, 78.50 U/L, and 324.17 U/L. The lignin concentrations corresponding to the top three enzyme activity of the three enzymes are all 2.5 g/L, 3.0 g/L, and 2.0 g/L respectively.

### Example 3

This example provides the influence of the initial pH value of the culture medium on the enzyme activities of lignin peroxidase (LiP), manganese peroxidase (MnP), and laccase (Lac) produced by the strain QL-Z3.

The strain QL-Z3 was inoculated into 50 mL of liquid LB medium (containing 50 mg/L ampicillin), cultured at 30° C. and 180 rpm for overnight. The bacterial solution was transferred to 2 mL EP tubes for freezing centrifugation and concentration, then was washed by sterilized saline water to remove the remaining LB, and suspended. The prepared liquid medium was adjusted to different pH values (5.0, 6.5, 8.0, 9.5, and 11.0), with 3 replicates. The bacterial suspension was inoculated into the lignin liquid fermentation medium for fermentation culture at 30° C., 180 rpm for 7 days. The fermentation broth was centrifuged every 24 hours, and the supernatant was collected to measure the enzyme activities of LiP, Lac, and MnP, and the enzyme activity change curves were plotted.

From FIGS. 4, 5, and 6, it can be seen that the three lignin degradation-related enzymes all shows enzyme activities in lignin liquid culture medium with different pH values, shows higher enzyme activities under alkaline conditions, and the enzyme activities of all three enzymes reached the highest at pH 9.5, where the lignin peroxidase (LiP) was 133.00 U/L, the laccase (Lac) was 236.00 U/L, and the manganese peroxidase (MnP) was 251.67 U/L.

### Example 4

This example provides the influence of different nitrogen sources on the enzyme activities of lignin peroxidase (LiP), laccase (Lac), and manganese peroxidase (MnP) produced by the strain QL-Z3.

The strain QL-Z3 was inoculated into 50 mL of liquid LB medium (containing 50 mg/L ampicillin), cultured at 30° C. and 180 rpm for overnight. The bacterial solution was transferred to 2 mL EP tubes for freezing centrifugation and concentration, then was washed by sterilized saline water to remove the remaining LB, and suspended. Using lignin as the sole carbon source and with a total nitrogen content of 2.0 g/L yeast powder as the standard, different nitrogen sources (peptone, yeast powder, NH₄NO₃, NaNO₃, (NH₄)₂SO₄) with the same nitrogen content were added to the liquid medium with three replicates. After inoculation, the inoculation solution was fermentation cultured at 30° C., 180 rpm for 7 days, and the fermentation broth was centrifuged every 24 hours and the supernatant was collected to measure the enzyme activities of LiP, Lac, and MnP, and the enzyme activity change curves were plotted.

During the nitrogen source optimization process, the amount of the used nitrogen sources was based on a total nitrogen content of 2.0 g/L yeast powder. From FIGS. 7, 8, and 9, it can be seen that the three lignin degradation-related enzymes all shows enzyme activities in lignin liquid culture medium with different nitrogen sources. The activities of the three enzymes added with organic nitrogen sources were significantly higher than those with inorganic nitrogen sources. When NH₄NO₃ was used as the nitrogen source, the activities of all three enzymes reached the highest, with the lignin peroxidase (LiP) activity being 251.50 U/L, the laccase (Lac) activity being 158.00 U/L, and the manganese peroxidase (MnP) activity being 204.38 U/L. The nitrogen sources that had the greatest impact on the three enzymes were NH₄NO₃, NaNO₃, and (NH₄)₂SO₄.

### Example 5

This example provides the influence of temperature on the enzyme activities of lignin peroxidase (LiP), laccase (Lac), and manganese peroxidase (MnP) produced by the strain QL-Z3.

The strain QL-Z3 was inoculated into 50 mL of liquid LB medium (containing 50 mg/L ampicillin), cultured at 30° C. and 180 rpm for overnight. Separately, the bacterial solutions were transferred to 2 mL EP tubes for freezing centrifugation and concentration, and then was washed by sterilized saline water to remove the remaining LB, and then suspended. The bacterial suspension was inoculated into lignin liquid culture mediums with different temperatures of 20° C., 25° C., 30° C., 35° C. and 40° C. for shake flask cultivation. The lignin liquid culture mediums, with 3 replicates, were placed and cultivated in the above-mentioned different temperature shakers at 180 rpm for 7 days. The fermentation broth was collected every 24 hours and centrifuged, then the supernatant was collected to measure the enzyme activities of LiP, MnP, and Lac, and the enzyme activity change curves were plotted.

From FIGS. 10, 11, and 12, it can be seen that the enzyme activities of laccase and manganese peroxidase reached the maximum enzyme activity at 30° C., which were 272.33 U/L and 238.75 U/L respectively. The lignin peroxidase reached the maximum enzyme activity of 109.50 U/L at 25° C., which was not much different from 101.50 U/L under 30° C. conditions. And the optimal cultivation temperature of strain QL-Z3 was 30° C. Therefore, in the orthogonal experiment, the cultivation temperature was no longer selected as the optimization condition.

### Example 6

This example provides the orthogonal test optimization scheme and optimization results for the enzyme activity of lignin peroxidase (LiP). After the optimization by the above examples, three levels of three factors namely lignin concentration (A), nitrogen source type (B), and initial pH of the culture medium (C), with significant differences, were selected to further optimize the enzyme activity. Table 5 shows the influencing factors and corresponding influencing levels. Table 6 is the test design table. Table 7 is the variance analysis of the orthogonal model.

TABLE 5
Influencing factors and influencing levels

| | Factors | | |
|---|---|---|---|
| | Lignin Concentration | Nitrogen Source | Culture Medium |
| Levels | (g · L⁻¹)(A) | Type(B) | pH(C) |
| 1 | 2 | NH₄NO₃ | 8 |
| 2 | 2.5 | NaNO₃ | 9.5 |
| 3 | 3 | (NH₄)₂SO₄ | 11 |

TABLE 6
Experimental Design Table

| | Lignin | | | |
|---|---|---|---|---|
| Test | Concentration | Nitrogen Source | Culture Medium | LiP Enzyme |
| Number | (g · L⁻¹)(A) | Type (B) | pH(C) | Activity(U/L) |
| 1 | 1(2.0) | 2(NaNO₃) | 3(11.0) | 164.50 |
| 2 | 1(2.0) | 3((NH₄)₂SO₄) | 2(9.5) | 218.40 |
| 3 | 1(2.0) | 1(NH₄NO₃) | 1(8.0) | 303.00 |
| 4 | 2(2.5) | 3((NH₄)₂SO₄) | 3(11.0) | 276.50 |
| 5 | 2(2.5) | 2(NaNO₃) | 1(8.0) | 264.50 |
| 6 | 2(2.5) | 1(NH₄NO₃) | 2(9.5) | 167.50 |
| 7 | 3(3.0) | 3((NH₄)₂SO₄) | 1(8.0) | 371.50 |
| 8 | 3(3.0) | 1(NH₄NO₃) | 3(11.0) | 210.50 |
| 9 | 3(3.0) | 2(NaNO₃) | 2(9.5) | 194.50 |
| K₁ | 685.90 | 681.00 | 939.00 | |
| K₂ | 708.50 | 623.50 | 580.40 | |
| K₃ | 776.50 | 866.40 | 651.50 | |
| k₁ | 238.63 | 303.67 | 273.00 | |
| k₂ | 302.83 | 174.50 | 226.80 | |
| k₃ | 225.50 | 288.80 | 267.17 | |
| Range R | 64.20 | 129.17 | 55.83 | |
| Optimal | A₃ | B₃ | C₁ | |
| Level | | | | |
| Primary and | B > A > C | | | |
| Secondary | | | | |
| Levels | | | | |
| Optimal | A₃B₃C₁ | | | |
| Combination | | | | |

Note:
K₁, K₂, and K₃ represent the sum of the corresponding LiP enzyme activities of 1, 2, and 3 respectively under a single factor condition. k₁, k₂, and k₃ respectively are the corresponding average values. R is the difference between the maximum k value and the minimum k value.

TABLE 7
Orthogonal Model Variance Analysis

| | Sum of | Degrees of | Mean | | | |
|---|---|---|---|---|---|---|
| Source | Squares | Freedom | Square | F Value | P Value | Significance |
| Correction | 54692.040^{a} | 6 | 9115.340 | 26.573 | 0.037 | * |
| Model | | | | | | |
| Intercept | 514041.201 | 1 | 514041.201 | 1498.512 | 0.001 | ** |
| A | 24321.236 | 2 | 12160.618 | 0.509 | 0.027 | * |
| B | 25026.202 | 2 | 12513.101 | 0.842 | 0.027 | * |
| C | 5344.602 | 2 | 2672.301 | 1.304 | 0.114 | |
| Error | 686.069 | 2 | 343.034 | | | |
| Total | 569419.310 | 9 | | | | |
| Corrected | 55378.109 | 8 | | | | |
| Total | | | | | | |
| Correlation | R² = 0.988(Adjusted to R² = 0.950) | | | | | |
| Coefficient | | | | | | |

Note:
** represents p < 0.01, indicating a highly significant difference;
* represents p < 0.05, indicating a significant difference.

The F value of the model is 26.573, and P value is less than 0.05, R2 is close to 1, indicating a high level of reliability in the analysis results and the model is significant, which can be used to predict the effects of the three factors on the activity of lignin peroxidase (LiP). The effects of lignin concentration (A) and nitrogen source type (B) on the enzyme activity of the strain LiP are significant. The influence of each factor on the enzyme activity can be determined by the corresponding range R value of the factors. The larger the R value, the stronger the influence on the result, and the ranking is: nitrogen source type (B)>lignin concentration (A)>culture medium pH (C).

Through experimental analysis, A₃B₃C₁ is the fermentation condition combination of the crude enzyme solution of lignin peroxidase, that is, pH is selected as 8, nitrogen source is NH₄NO₃, and lignin concentration is 3 g/L. Under these conditions, the enzyme activity of LiP can be optimized to 371.00 U/L, which is 3.53 times that of before optimization, and the optimization result is significant. After four repetitive experiments, the LiP enzyme activity of the optimal combination is measured as: 385.00 U/L, 350.00 U/L, 321.00 U/L, 417.00 U/L, with an average value of 368.30 U/L. The experimental results have repeatability, so the experimental results are valid.

### Example 7

This example provides the orthogonal test optimization results of laccase (Lac). Table 8 is the test design table. Table 9 is the orthogonal model variance analysis.

TABLE 8
Test Design Table

| | | Nitrogen | | |
|---|---|---|---|---|
| Test | Lignin Concentration | Source Type | Culture Medium | LiP Enzyme |
| Number | (g · L⁻¹)(A) | (B) | pH(C) | Activity(U/L) |
| 1 | 1(2.0) | 2(NaNO₃) | 3(11.0) | 190.50 |
| 2 | 1(2.0) | 3((NH₄)₂SO₄) | 2(9.5) | 149.00 |
| 3 | 1(2.0) | 1(NH₄NO₃) | 1(8.0) | 159.00 |
| 4 | 2(2.5) | 3((NH₄)₂SO₄) | 3(11.0) | 167.00 |
| 5 | 2(2.5) | 2(NaNO₃) | 1(8.0) | 135.44 |
| 6 | 2(2.5) | 1(NH₄NO₃) | 2(9.5) | 219.00 |
| 7 | 3(3.0) | 3((NH₄)₂SO₄) | 1(8.0) | 59.17 |
| 8 | 3(3.0) | 1(NH₄NO₃) | 3(11.0) | 158.67 |
| 9 | 3(3.0) | 2(NaNO₃) | 2(9.5) | 151.33 |
| K₁ | 498.50 | 536.67 | 353.61 | |
| K₂ | 521.44 | 477.27 | 519.33 | |
| K₃ | 369.17 | 375.17 | 516.17 | |
| k₁ | 166.17 | 178.89 | 117.87 | |
| k₂ | 173.81 | 159.09 | 173.11 | |
| k₃ | 123.06 | 125.06 | 172.06 | |
| Range R | 50.76 | 53.83 | 55.24 | |
| Optimal | A₂ | B₁ | C₂ | |
| Level | | | | |
| Primary and | C > B > A | | | |
| Secondary | | | | |
| Levels | | | | |
| Optimal | A₂B₁C₂ | | | |
| Combination | | | | |

Note:
K₁, K₂, and K₃ represent the sum of Lac enzyme activities at levels 1, 2, and 3 respectively under a single factor. k₁, k₂, and k₃ respectively are the corresponding average values. R is the difference between the maximum k value and the minimum k value.

TABLE 9
Orthogonal Model Variance Analysis

| | Sum of | Degrees of | | | | |
|---|---|---|---|---|---|---|
| Source | Squares | Freedom | Mean Square | F Value | P Value | Significance |
| Correction | 14930.280^{a} | 6 | 2488.380 | 23.583 | 0.041 | * |
| model | | | | | | |
| Intercept | 214402.955 | 1 | 214402.955 | 2031.916 | 0.000 | ** |
| A | 4493.183 | 2 | 2246.591 | 21.291 | 0.045 | * |
| B | 4448.336 | 2 | 2224.168 | 21.079 | 0.045 | * |
| C | 5988.762 | 2 | 2994.381 | 28.378 | 0.034 | * |
| Error | 211.035 | 2 | 105.518 | | | |
| Total | 229544.270 | 9 | | | | |
| Corrected | 15141.316 | 8 | | | | |
| Total | | | | | | |
| Correlation | R² = 0.986(Adjusted to R² = 0.944) | | | | | |
| Coefficient | | | | | | |

Note:
*** represents p < 0.01, indicating a highly significant difference;
* represents p < 0.05, indicating a significant difference.

From Table 9, it can be seen that F value of the model is 23.583, and P value is less than 0.05, R² is close to 1, which indicates that the result is highly reliable, the model is significant, and can be used to predict the effects of these three factors on the activity of laccase. In the analysis results of the orthogonal combination variance model, the influence of lignin concentration (A), nitrogen source type (B), and initial culture medium pH (C) on the enzyme activity of Lac produced by the strain in the degradation of lignin is significant. According to the corresponding range R value of each factor, the influence of each factor on the degradation situation can be determined, and the larger the R value, the stronger the influence on the result, and the ranking is: culture medium pH (C)>nitrogen source type (B)>lignin concentration (A).

After experimental analysis, A₂B₁C₂ is the optimal combination of fermentation conditions for laccase, that is, pH is selected as 9.5, the nitrogen source is NH₄NO₃, and the lignin concentration is 2.5 g/L. At this time, the enzyme activity of Lac can be optimized to 219.00 U/L, which is 2.84 times that of before optimization, and the optimization result is significant. After four confirmatory experiments, the Lac enzyme activity results of the optimal combination are measured as: 220.00 U/L, 215.00 U/L, 222.00 U/L, 226.00 U/L, with an average value of 220.80 U/L. The experimental results have repeatability, so the experimental results are valid.

### Example 8

This example presents the orthogonal test optimization results of manganese peroxidase (MnP). Table 10 is the test design table. Table 11 is the orthogonal model variance analysis.

TABLE 10
Test Design Table

| | | | Culture | |
|---|---|---|---|---|
| Test | Lignin Concentration | Nitrogen Source | Medium | MnP Enzyme |
| Number | (g · L⁻¹)(A) | [[Type]] (B) | pH(C) | Activity(U/L) |
| 1 | 1(2.0) | 2(NaNO₃) | 3(11.0) | 245.00 |
| 2 | 1(2.0) | 3((NH₄)₂SO₄) | 2(9.5) | 470.83 |
| 3 | 1(2.0) | 1(NH₄NO₃) | 1(8.0) | 728.75 |
| 4 | 2(2.5) | 3((NH₄)₂SO₄) | 3(11.0) | 396.67 |
| 5 | 2(2.5) | 2(NaNO₃) | 1(8.0) | 650.50 |
| 6 | 2(2.5) | 1(NH₄NO₃) | 2(9.5) | 839.50 |
| 7 | 3(3.0) | 3((NH₄)₂SO₄) | 1(8.0) | 338.75 |
| 8 | 3(3.0) | 1(NH₄NO₃) | 3(11.0) | 285.00 |
| 9 | 3(3.0) | 2(NaNO₃) | 2(9.5) | 443.44 |
| K₁ | 1444.58 | 1853.25 | 1718.00 | |
| K₂ | 1886.67 | 1338.94 | 1753.77 | |
| K₃ | 1067.19 | 1206.25 | 926.67 | |
| k₁ | 481.53 | 617.75 | 572.67 | |
| k₂ | 628.89 | 446.31 | 584.59 | |
| k₃ | 355.73 | 402.08 | 308.89 | |
| Range R | 273.16 | 215.67 | 275.70 | |
| Optimal | A₂ | B₁ | C₂ | |
| Level | | | | |
| Primary and | C > A > B | | | |
| Secondary | | | | |
| Levels | | | | |
| Optimal | A₂B₁C₂ | | | |
| Combination | | | | |

Note:
K₁, K₂, and K₃ represent the sum of the MnP enzyme activities at levels 1, 2, and 3 respectively under a single factor. k₁, k₂, and k₃ respectively are the corresponding average values. R is the difference between the maximum k value and the minimum k value.

TABLE 11
Orthogonal Model Variance Analysis

| | | Degrees | | | | |
|---|---|---|---|---|---|---|
| | Sum of | of | Mean | | | |
| Source | Squares | Freedom | Square | F Value | P Value | Significance |
| Correction | 335746.859^{a} | 6 | 55957.810 | 21.026 | 0.046 | * |
| model | | | | | | |
| Intercept | 2149586.048 | 1 | 2149586.048 | 807.701 | 0.001 | ** |
| A | 112157.139 | 2 | 56078.569 | 21.071 | 0.045 | * |
| B | 77858.935 | 2 | 38929.467 | 14.628 | 0.064 | |
| C | 145730.786 | 2 | 72865.393 | 27.379 | 0.035 | * |
| Error | 5322.729 | 2 | 2661.364 | | | |
| Total | 2490655.636 | 9 | | | | |
| Corrected | 341069.588 | 8 | | | | |
| Total | | | | | | |

| | |
|---|---|
| Correlation | R² = 0.984(Adjusted to R² = 0.938) |
| Coefficient | |

Note:
** represents p < 0.01, indicating a highly significant difference;
* represents p < 0.05, indicating a significant difference.

From Table 11, it can be seen that the F value of the model is 21.026, P value is less than 0.05, and R² is close to 1. The reliability of this result is high, and the model is significant, which can be used to predict the effects of these three factors on the enzyme activity of manganese peroxidase. The effects of lignin concentration (A) and initial pH of the culture medium (C) on the manganese peroxidase (MnP) of the strain are significant. The influence of each factor on the degradation situation can be determined by the corresponding range R value of each factor. The larger the R value, the stronger the influence on the result, and the ranking is: initial pH of the culture medium (C)>lignin concentration (A)>type of nitrogen source (B).

After the experimental analysis, A₂B₁C₂ is the optimal combination of fermentation conditions for manganese peroxidase (MnP), that is, pH is selected as 9.5, the nitrogen source is NH₄NO₃, and the lignin concentration is 2.5 g/L. The enzyme activity of manganese peroxidase can be optimized to 839.50 U/L, which is 3.18 times that of before optimization. The optimization result is significant. After 4 confirmatory experiments, the enzyme activity results of MnP under this optimal combination are measured as: 815.00 U/L, 802.50 U/L, 815.00 U/L, 792.50 U/L, with an average value of 806.10 U/L. The experimental results have repeatability, so the experimental results are valid.

The above-mentioned embodiments are part of the embodiments of the present disclosure, rather than all the embodiments. The detailed description of the embodiments of the present disclosure is not intended to limit the scope of protection of the present disclosure, but merely represents the selected embodiments of the present disclosure. All other embodiments derived through relevant deductions and substitutions by ordinary technicians in the field based on the concept of the present disclosure, without making any creative efforts, are all within the scope of protection of the present disclosure.

What is claimed is:

1. A method for enhancing degradation performance of lignin degradation bacteria, wherein the lignin degradation bacteria are Erwinia sp. QL-Z3 strain, and activated Erwinia sp. QL-Z3 strain are inoculated into lignin liquid medium at 30° C. and 180 rpm for cultivation;
pH of the lignin liquid medium is 5, nitrogen source is (NH₄)₂SO₄, and lignin addition amount is 1.5 g/L;
components and contents of each 1 L of the lignin liquid medium are: 1.5 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of (NH₄)₂SO₄.
degradation rate of the lignin by the Erwinia sp. QL-Z3 strain is optimized to 25.24%;
by adjusting pH of the lignin liquid medium to 8, nitrogen source to NH₄NO₃, and lignin addition amount to 3 g/L, enzyme activity of the lignin peroxidase in the Erwinia sp. QL-Z3 strain is optimized to 371.00 U/L;
by adjusting pH of the lignin liquid medium to 9.5, the nitrogen source to NH₄NO₃, and the lignin addition amount to 2.5 g/L, the enzyme activity of the laccase in the Erwinia sp. QL-Z3 strain is optimized to 219.00 U/L;
by adjusting the pH of the lignin liquid medium to 9.5, the nitrogen source to NH₄NO₃, and the lignin addition amount to 2.5 g/L, the enzyme activity of the manganese peroxidase in the Erwinia sp. QL-Z3 strain is optimized to 839.50 U/L;
the GenBank accession number of 16SrRNA gene in the Erwinia sp. QL-Z3 strain is MH828331, and the GenBank accession number of the whole gene sequencing is CP037950.

2. A method for enhancing enzyme activity of lignin peroxidase in lignin degradation bacteria, wherein Erwinia sp. QL-Z3 strain of the lignin degradation bacteria are cultivated in lignin liquid medium at 30° C. and 180 rpm, and the enzyme activity of the lignin peroxidase in the Erwinia sp. QL-Z3 strain is optimized to 371.00 U/L;
the medium is a lignin liquid medium, pH of the lignin liquid medium is 8, nitrogen source is NH₄NO₃, and lignin addition amount is 3 g/L; components and contents of each 1 L of the lignin liquid medium are: 3 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of NH₄NO₃;
the GenBank accession number of 16SrRNA gene in the Erwinia sp. QL-Z3 strain is MH828331, and the GenBank accession number of the whole gene sequencing is CP037950.

3. A method for enhancing enzymatic activity of laccase in lignin degradation bacteria, wherein Erwinia sp. QL-Z3 strain of the lignin degradation bacteria are cultivated in lignin liquid medium at 30° C. and 180 rpm, and the enzymatic activity of the laccase in the Erwinia sp. QL-Z3 strain is optimized to 219.00 U/L;
the medium is a lignin liquid medium, with a pH of 9.5, nitrogen source of NH₄NO₃, and lignin addition of 2.5 g/L; components and contents of each 1 L of the lignin liquid medium are: 2.5 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of NH₄NO₃;
the GenBank accession number of 16SrRNA gene in the Erwinia sp. QL-Z3 strain is MH828331, and the GenBank accession number of the whole gene sequencing is CP037950.

4. A method for enhancing enzymatic activity of manganese peroxidase in lignin degradation bacteria, wherein Erwinia sp. QL-Z3 strain of the lignin degradation bacteria are cultivated in lignin liquid medium at 30° C. and 180 rpm, and the enzymatic activity of manganese peroxidase in Erwinia sp. QL-Z3 strain are optimized to 839.50 U/L;
the medium is a lignin liquid medium, with a pH of 9.5, nitrogen source of NH₄NO₃, and lignin addition of 2.5 g/L; the composition and content of each 1 L of the lignin liquid medium are: 2.5 g of Lignin, 2 g of K₂HPO₄, 0.3 g of MgSO₄·7H₂O, 0.08 g of CaCl₂, 0.05 g of FeSO₄·7H₂O, 0.02 g of MnCl₂, 2 g of NH₄NO₃;
the GenBank accession number of 16SrRNA gene in the Erwinia sp. QL-Z3 strain is MH828331, and the GenBank accession number of the whole gene sequencing is CP037950.
